(19) United States
(12) Patent Application Publication
(10) Pub. No.: US 2012/0116463 A1
(43) Pub. Date: May 10, 2012

(54) SYSTEM FOR OSTEOSYNTHESIS, IN PARTICULAR FOR VERTEBRAL OSTEOSYNTHESIS

(76) Inventor: Hassan Razian, L'Hay les Roses (FR)

(21) Appl. No.: 13/386,101

(22) PCT Filed: Jul. 19, 2010
(86) PCT No.: PCT/FR2010/000514

§ 371 Date: Jan. 20, 2012

(30) Foreign Application Priority Data
Jul. 22, 2009 (FR) 09 03607

(51) Int. Cl. A61B 17/86

(52) U.S. Cl. 606/305

(57) ABSTRACT

An osteosynthesis system includes a pedicular screw and a bar of longitudinal axis, the screw having a U-shaped head with two branches and a base for defining an inter-branch space in which the bar is received, a screw shank with a bone thread, and elements for blocking the bar in the space and including at least a tab and movement elements for moving the tab relative to the branches in a plane substantially parallel to the axis, the movement elements being arranged so that the tab occupies two positions, a first position where the tab does not co-operate with the bar, and a second position where the tab co-operates with at least one of the branches in such a manner that at least a portion of the tab is situated in the space and applies on the bar a pressure force that is directed towards the base of the U-shape.

The present invention relates to osteosynthesis systems that find a particularly advantageous application in spinal osteosynthesis.

Osteosynthesis systems, in particular spinal systems, are already known that comprise at least one pedicular screw and at least one bar defined along a longitudinal axis; the pedicular screw having a U-shaped head with two branches and a base uniting the two branches so as to define an inter-branch space, the distance between the two branches being not less than the greatest transverse thickness of the bar, so that the bar is suitable for being received in the inter-branch space; a screw shank having a penetrating first end and a bone thread on its outside surface; means for coupling the second end of the screw shank opposite from its penetrating end with the base of the head; and means for blocking the bar in the inter-branch space.

In the prior art, as illustrated in particular by US 2005/049589, US 2005/277925, and US 2003/187433, the means for blocking the bar in the inter-branch space are not very convenient for the practitioner to put into place, since they include a nut or the like for screwing either around the two branches or else between the two branches.

Thus, an object of the present invention is to provide an osteosynthesis system, in particular for the spine, that mitigates the above-mentioned drawbacks of prior art systems, at least to a large extent, i.e. that can make the work of the practitioner much easier while performing surgery, in particular by making it easier and quicker to block the bar between the two branches of the U-shape, mainly for the purpose of reducing the duration of the surgery while also ensuring that the blocking is safe, with this being for the best possible patient well-being.

More precisely, the present invention provides an osteosynthesis system including at least one pedicular screw and at least one bar defined along a longitudinal axis, and as defined in accompanying claim 1.

Other characteristics and advantages of the present invention appear from the following description given with reference to the accompanying drawings that are purely illustrative and non-limiting, and in which:

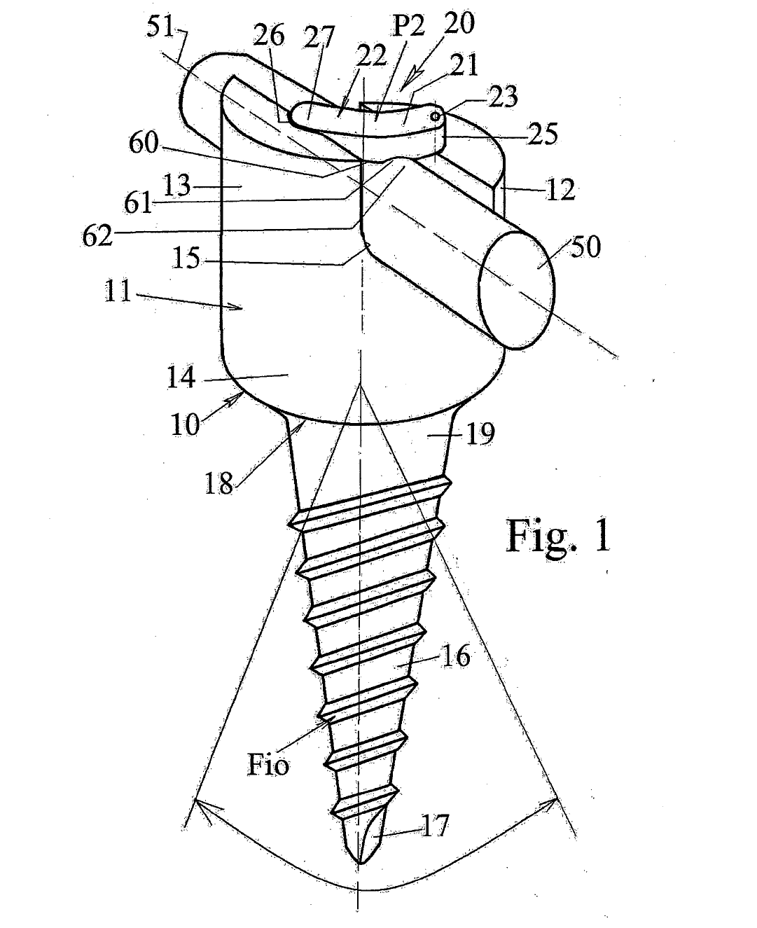

FIG. 1 is an isometric perspective view of a first embodiment of the osteosynthesis system of the invention;

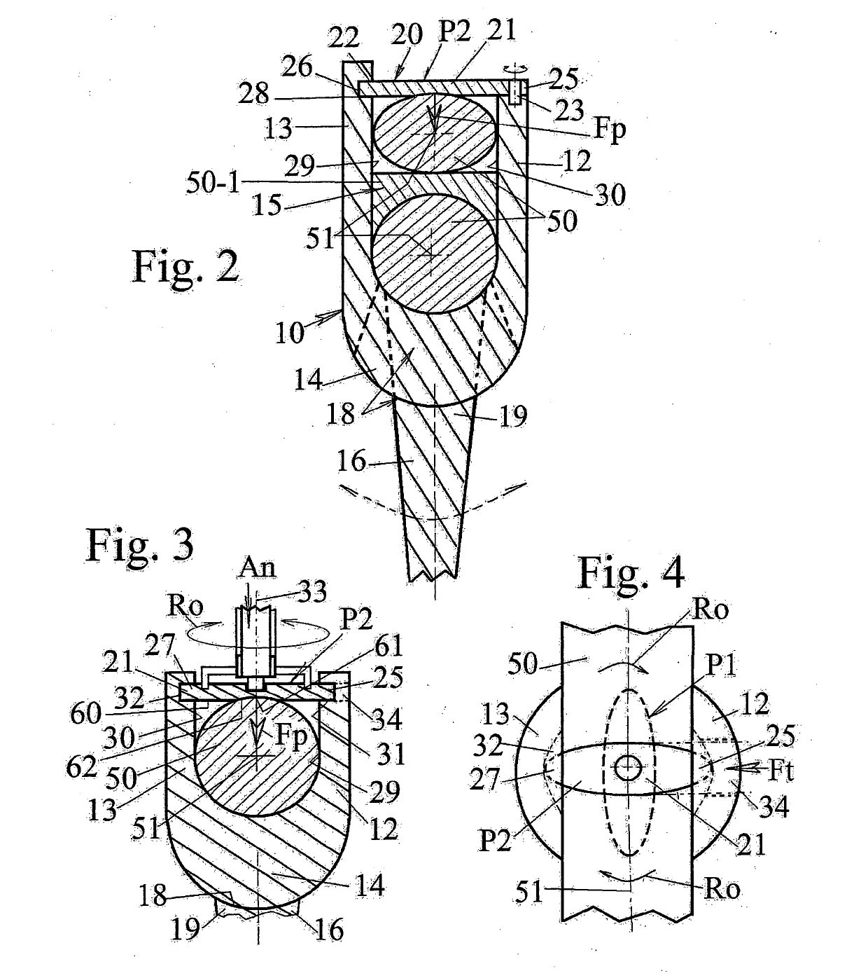

FIG. 2 is a longitudinal section view of the embodiment of the osteosynthesis system shown in FIG. 1, but showing a different embodiment of the means for blocking one of the elements, in particular the tab;

FIGS. 3 and 4 both show simultaneously second and third embodiments of the osteosynthesis system of the invention, FIG. 3 being a diagrammatic longitudinal section through both embodiments, and FIG. 4 being a plan view; and

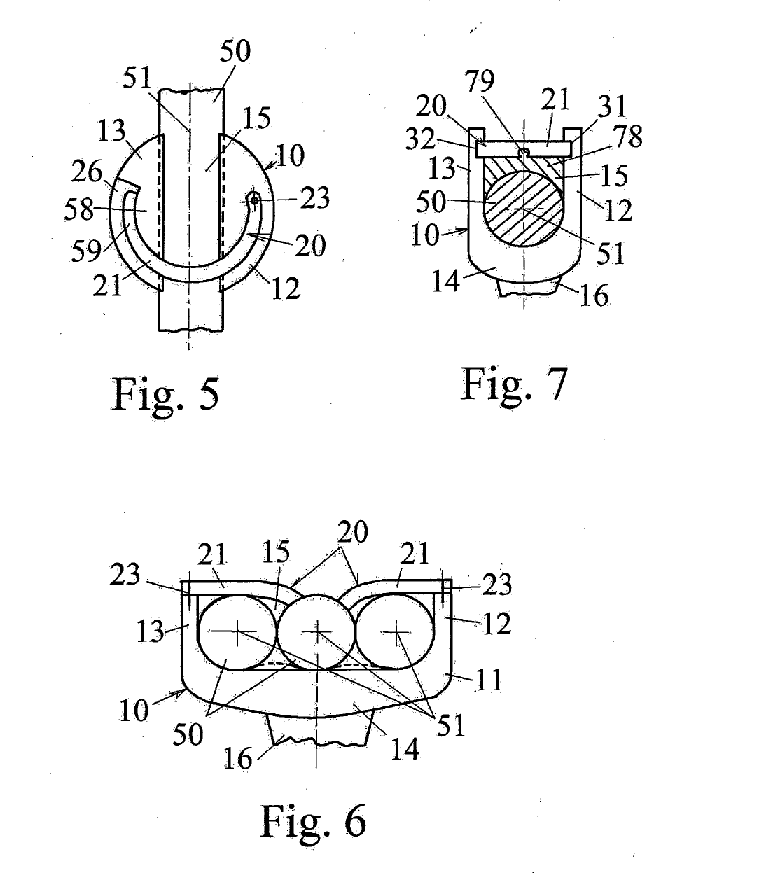

FIGS. 5 to 7 show variant embodiments of the osteosynthesis system of the invention as shown in FIGS. 1 to 4, FIGS. 5 and 6 showing more particularly variants of the embodiments of FIGS. 1 and 2, while FIG. 7 is more particularly a variant of one of the embodiments of FIGS. 3 and 4.

With reference to the accompanying figures, the present invention relates to an osteosynthesis system that finds a particularly advantageous application as a system for spinal osteosynthesis, and that includes at least one pedicular screw 10 and at least one bar 50 defined along a longitudinal axis 51.

The pedicular screw 10 has a U-shaped head 11 that is often referred to by the person skilled in the art as a “tuning fork”, the head comprising two branches 12 and 13 together with a base 14 uniting the two branches and defining an inter-branch space 15 between them, the distance between the two branches being not less than the greatest transverse thickness of the bar 50, such that the bar is suitable for being received in the inter-branch space 15. The pedicular screw also includes a screw shank 16 having a penetrating first end 17 that is advantageously self-tapping and that carries a bone-type thread Fio on its outside surface, together with means 18 for coupling the second end 19 of the screw shank 16 that is opposite from its penetrating end 17 with the base 14 of the head 11. Possible embodiments of the coupling means 18 are given below.

Finally, the pedicular screw includes means 20 for blocking the bar 50 in the inter-branch space 15.

As defined below, the osteosynthesis system must include at least one bar 50. In the context of the invention, it may therefore include a plurality of such bars, e.g. two as in the example shown in FIG. 2.

It is even possible for spacer elements such as the element 50-1 also to be present. Consequently, in the meaning of the present invention, when reference is made to “blocking” a bar, the blocking may be obtained either by acting directly on the bar (FIGS. 1 and 3), or else indirectly by acting on one or another of the bars and/or on one or another of the spacer elements.

According to an essential characteristic of the invention, the means 20 for blocking the bar in the inter-branch space comprise at least one tab 21 and means for moving the tab 21 relative to the two branches 12 and 13 in a plane that is substantially parallel to the longitudinal axis 51. These means for moving the tab 21 are arranged in such a manner that the tab is suitable for occupying at least two positions P1 and P2, namely: a first position P1 (FIG. 4) in which the tab does not co-operate with the bar 50; and a second position P2 (FIGS. 1 to 7) in which the tab co-operates with at least one of the branches 12 and 13 in such a manner that at least a portion of the tab 21 is also situated in the inter-branch space 15, and bears directly or indirectly against the bar 50 with a pressure force Fp (FIG. 2) that is directed towards the base 14 of the U-shape.

In an advantageous first embodiment, the means for moving the tab 21 relative to the two branches 12, 13 in a plane that is substantially parallel to the longitudinal axis 51, and preferably perpendicular to the axis of the screw shank 16, are constituted by a pivot 23 of any type, e.g. a pivot pin, a ball joint, etc., defining an axis of rotation that is substantially orthogonal to the longitudinal axis 51, means for mounting the pivot so that it co-operates with a first one of the two branches 12 and 13, specifically the branch given reference 12 in all of the figures, and means for mounting a first end 25 of the tab 21 in pivoting co-operation with the pivot 23 so that the tab 21 is suitable for occupying its above-defined first and second positions P1 and P2 by pivoting.

The pivot may be made in other ways, for example by a rotary shaft, a screw forming a shaft that can end up by being blocked once the tab 21 is in its second position P2, or indeed in the form of a helical shaft or the like that itself becomes blocked in its bearing when the tab 21 goes from its first position P1 to its second position P2.

The osteosynthesis system of the invention preferably further includes means for at least temporarily blocking the tab 21 when it is in its second position P2.

In a preferred possible first embodiment, these at least temporarily blocking means for blocking the tab 21 when it is in its second position P2 are constituted by at least one notch 26 of concave shape, three of them being shown in FIG. 1 made in the other branch 13 so as to receive the second end 27 of the tab 21 when it is in its second position P2, by snap-fastening so as to enable it to conserve this second position P2, the length of the tab being determined so that it plunges forcibly into the notch by making use of the elastic deformation that is inherent to any material.

The notch 26 may be made in the surface of the second branch 13, as shown in FIG. 1, or in the bottom of a first groove 28 formed in the wall 29 of the second branch 13 and facing towards the inter-branch space 15, FIG. 2.

The plurality of notches 26, as shown in FIG. 1, enables different types of blocking to be obtained, starting from light, temporary blocking, to blocking that is more intense.

The above-described embodiment may be made in accordance with a variant as shown in FIG. 5. In this variant, the temporary blocking means of the tab 21 are constituted by the fact that the tab is in the shape of a hook 59 suitable for hook engagement with a complementary portion 58 formed on the second branch 13, it being emphasized that the complementary portion may be a portion of the secondary branch 13 itself.

This complementary portion 58 is also advantageously arranged so that the co-operation between the tab in the form of a hook 59 and the complementary portion 58 causes the two branches 12 and 13 to move towards each other so as to clamp the bar more tightly, as represented diagrammatically in FIG. 5.

In a second embodiment such as that shown in FIGS. 3, 4, and 7, the means for moving the tab 21 relative to the two branches 12 and 13 in a plane substantially parallel to the longitudinal axis 51 are constituted by second and third grooves 31 and 32 made facing each other in the two facing walls 29 and 30 of the two branches 12 and 13, and by the fact that the tab 21 is constituted by a part of oblong shape, said part having: i) a length that is greater than the distance between the two facing walls and no greater than the distance between the bottoms of the two facing walls; ii) a thickness no greater than the width of the second and third grooves; and iii) a width that is less than the distance between the two facing walls.

These characteristics make it possible firstly to position the tab 21 in the inter-branch space 15 in its first position P1, FIG. 4, substantially parallel with the two branches 12 and 13, and then to cause it to turn, Ro, about an axis 33 perpendicular to the longitudinal axis 51 so that its two ends 25 and 27 are inserted respectively into the second and third grooves 31 and 32.

By way of example, the turning, Ro, is obtained by an instrument An or the like shown diagrammatically in FIG. 3 and suitable for turning the tab 21 while exerting forces on its two ends 25 and 27 so as to make them penetrate into respective ones of the two grooves 31 and 32 so that the tab can exert the pressure force Fp on the bar 50.

The above-described embodiment may be made in accordance with a variant like that shown in FIG. 7. In this variant, the means 20 for blocking the bar 50 in the inter-branch space 15 further include an insert 78 adapted to be interposed between the tab 21 and the bar 50, and means 79 for mounting the insert 78 on the tab 21 so that it is free to rotate, e.g. by free clip-fastening or crimping, or the like.

This variant embodiment enables the tab 21 to be properly guided while it is being inserted into the inter-branch space 15, with the bar 50 having the insert 78 placed astride it, as shown, and thereafter, once the tab 21 has been pivoted in the manner described above, this variant makes it possible to distribute all of the forces on the bar 50, thereby even better holding the bar in the inter-branch space 15.

In a third embodiment of the osteosynthesis system, the means 20 for moving the tab 21 relative to the two branches 12 and 13 in a plane substantially parallel to the longitudinal axis 51 are constituted, still with reference to FIGS. 3 and 4 which in fact show two possible embodiments, by a fourth groove 32 that is the same as the third groove mentioned in the above description of the second embodiment, which groove is made in a second branch 13 of the two branches 12 and 13, this fourth groove 32 opening out into the inter-branch space 15, and a through hole 34 made in the other branch 12, i.e. the first branch, and opening out into the inter-branch space 15 substantially facing the fourth groove 32.

Under such circumstances, the tab 21 is constituted by a part of oblong shape and it presents the following structural characteristics: i) a length greater than the distance between the two facing walls of the two branches 12 and 13; and ii) a thickness no greater than the smaller of the widths respectively of the fourth groove 32 and of the through hole 34.

These characteristics enable the tab 21 to take up its second position P2 as follows: it is initially inserted in the through hole 34 and then moved in translation along arrow Ft so as to be engaged in the fourth groove 32 and exert the pressure force Fp on the bar 50, the force being directed towards the base 14 of the U-shape and being produced essentially by elastic reaction of the material of the tab 21 against the two branches 12 and 13 with which it co-operates while being moved in translation Ft.

Advantageously, and regardless of the three above-described embodiments, the face 60 of the tab 21 via which the tab bears directly against a surface portion 62 of the bar 50 while it is in its second position P2, thereby pressing the bar 50 with pressure force Fp directed towards the base 14 of the U-shape, includes a groove 61 of shape substantially complementary to the surface portion 62 of the bar 50 so that the groove 61 is suitable for fitting closely to this surface portion 62, thereby contributing to holding the bar 50 securely while adding additional temporary blocking of the tab in its second position P2.

As mentioned above, the osteosynthesis system includes means 18 for coupling the second end 19 of the screw shank 16 with the base 14 of the head 11. These coupling means 18 may be of various types. Nevertheless, depending on the requirements of the practitioner who is going to use the osteosynthesis system, they are preferably selected from one of the three following means: i) fastening the second end 19 of the screw shank 16 to the base of the head 11, e.g. by welding, screw-fastening, etc.; ii) making the head 11 and the screw shank 16 as a single piece; and iii) mutual male-female spherical engagement between a spherical male portion secured to the second end 19 of the screw shank and a complementary spherical female portion formed in the bottom of the inter-branch space 15, the screw shank 16 passing through the base 14 via an orifice of section greater than the section of the screw shank and less than the section of the male spherical portion, and the male spherical portion being pivotally mounted in the female spherical portion.

These third means iii) are shown diagrammatically in FIG. 2, in part using dashed lines, the bottom circle possibly representing the cross-section of a bar 50 or else representing the section of the above-defined spherical male portion. This embodiment enables the head 11 to be steered relative to the screw shank 16, in particular when the shank is already implanted in a bony medium.

FIG. 6 shows a variant of the embodiment of the osteosynthesis system shown in FIGS. 1, 2, and 5.

This variant embodiment serves to show that the first branch 12 may, in certain circumstances, constitute the second branch 13, and vice versa. The figure also shows that the tab 21 secured to a branch 12, 13 may extend into the inter-branch space 15 without reaching the other branch 13, 12. It also shows that the osteosynthesis system may have a plurality of bars 50 arranged in the inter-branch space 15, not one above another as shown in FIG. 2, all lying in a plane that is substantially parallel to the axis of the screw shank 16, but rather juxtaposed one beside another in a plane that is substantially perpendicular to the axis. Furthermore, this embodiment shows that the tab 21 may pass entirely over the bar 50 that it is to block, or it may bear thereagainst solely via its end.

1. An osteosynthesis system including at least one pedicular screw (10) and at least one bar (50) defined along a longitudinal axis (51), said pedicular screw (10) comprising:
a U-shaped head (11) having two branches (12, 13) and a base (14) uniting the two branches and defining an inter-branch space (15), the distance between the two branches being not less than the greatest transverse thickness of said bar (50) so that said bar is suitable for being received in said inter-branch space (15);
a screw shank (16) having a penetrating first end (17) with a bone thread (Fio) on its outside surface;
means (18) for coupling the second end (19) of the screw shank (16) opposite from its penetrating end (17) with the base (14) of the head (11); and
means (20) for blocking said bar (50) in said inter-branch space (15), said means comprising at least one tab (21), movement means for moving said tab (21) relative to said two branches (12, 13) in a plane that is substantially parallel to said longitudinal axis (51), said movement means being arranged in such a manner that said tab is suitable for occupying at least two positions (P1, P2), a first position (P1) in which said tab does not co-operate with said bar (50), and a second position (P2) in which said tab co-operates with at least one of said branches (12, 13) in such a manner that at least a portion of said tab (21) is situated in said inter-branch space (15) and applies on said bar (50) a pressure force (Fp) directed towards the base (14) of the U-shape;
the system being characterized by the fact that the means for moving said tab (21) relative to said two branches (12, 13) are constituted by:
a pivot (23) defining a pivot axis that is substantially orthogonal to said longitudinal axis (51);
means for mounting said pivot in co-operation with a first branch (12) of the two branches (12, 13); and
means for mounting a first end (25) of said tab (21) in pivoting co-operation with said pivot (23) in such a manner that said tab (21) is suitable for occupying said first and second positions by turning.

2. A system according to claim 1, characterized by the fact that it further includes means for temporarily blocking said tab (21) when it is in its second position (P2).

3. A system according to claim 2, characterized by the fact that the means for temporarily blocking said tab (21) are constituted by a notch (26) formed in the other or “second” branch (13), said notch being made in such a manner as to receive the second end (27) of said tab (21) when it is in its second position (P2).

4. A system according to claim 3, characterized by the fact that said notch (26) is made in a first groove (28) made in the wall (29) of said second branch (13) and facing towards the inter-branch space (15).

5. A system according to claim 2, characterized by the fact that the means for temporarily blocking said tab (21) are constituted by the fact that said tab (21) is in the shape of a hook (59) suitable for co-operating by hook engagement with a complementary portion (58) formed on the second branch (13).

6. A system according to claim 1, characterized by the fact that it further includes second and third grooves (31, 32) made facing each other in the two facing walls (29, 30) of the two branches (12, 13), and by the fact that said tab (21) is constituted by a part of oblong shape, said part having a length greater than the distance between said two facing walls and no greater than the distance between the bottoms of the two facing grooves, a thickness no greater than the width of said second and third grooves, and a width less than the distance between the two facing walls.

7. A system according to claim 6, characterized by the fact that the means (20) for blocking said bar in said inter-branch space further comprise an insert (78) suitable for being interposed between said tab (21) and said bar (50), and means (79) for mounting said insert (79) in rotation on said tab (21).

8. A system according to claim 1, characterized by the fact that the face (60) of said tab (21), whereby said tab comes to bear directly on a surface portion (62) of said bar (50) when in its second position (P2) and applying on said bar (50) a pressure force (Fp) directed towards the base (14) of the U-shape, includes a groove (61) of shape substantially complementary to said surface portion (62) of said bar (50).

9. A system according to claim 1, characterized by the fact that the means (18) for coupling the second end (19) of the screw shank (16) with the base (14) of said head (11) are constituted by one of the three following means: fastening the second end (19) of the screw shank (16) on the base of the head (11); making the head (11) and the screw shank (16) as a single piece; and male-female spherical engagement between a spherical male portion secured to the second end (19) of the screw shank and a complementary spherical female portion made in the bottom of the inter-branch space (15), said screw head (16) passing through said base (14) via an orifice of circular section greater than the section of the screw head and less than the section of the male spherical portion, said spherical male portion being pivotally mounted in said spherical female portion.

10. A system according to claim 1, characterized by the fact that it constitutes at least a portion of a system for spinal osteosynthesis.
